(19) United States
(12) Patent Application Publication
(10) Pub. No.: US 2002/0107275 A1
(43) Pub. Date: Aug. 8, 2002

(54) Novel crystal and solvate forms of ondansetron hydrochloride and processes for their preparation

(76) Inventors: Ramy Lidor-Hadas, Kfar Saba (IL); Judith Aronhime, Rehovot (IL); Revital Lifshitz, Herzlia (IL); Shlomit Weizel, Petah Tikva (IL); Valerie Niddam, Even-Yeouda (IL); Asher Maymon, Petach Tikva (IL)

Correspondence Address: KENYON & KENYON, ONE BROADWAY, NEW YORK, NY, 10004, US

(21) Appl. No.: 10/016,752

(22) Filed: Oct. 30, 2001

Related U.S. Application Data
(60) Provisional application No. 60/244,283, filed on Oct. 30, 2000.
(60) Provisional application No. 60/253,819, filed on Nov. 29, 2000.
(60) Provisional application No. 60/265,539, filed on Jan. 31, 2001.

(51) Int. Cl. A61K 31/4178; C 07D 4 3/02

(52) U.S. Cl. 514/397; 548/311.4

(57) ABSTRACT

The present invention provides novel ondansetron hydrochloride crystalline polymorphic forms and solvates. Processes for making and interconverting the polymorphic forms are also provided. Further provided are pharmaceutical compositions and therapeutic methods using the novel polymorphic forms and hydrates.

### CROSS-REFERENCE TO RELATED APPLICATION

This application claims the benefit of provisional application serial No. 60/244,283, filed Oct. 30, 2000; provisional application serial No. 60/253,819, filed Nov. 29, 2000 and provisional application serial No. 60/265,539, filed Jan. 31, 2001.

### FIELD OF THE INVENTION

The present invention relates to novel polymorphic forms and hydrates of ondansetron hydrochloride and methods of making polymorphic and hydrate forms of ondansetron hydrochloride.

### BACKGROUND OF THE INVENTION

(±)1,2,3,9-Tetrahydro-9-methyl-3-[2-methyl-1h-imidazol-1-yl)methyl]-4h-carbazol-4-one having the molecular structure

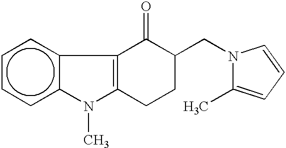

is a selective 5-HT₃ receptor antagonist. It is known by the generic name ondansetron. Ondansetron reduces nausea in patients undergoing chemotherapy. Grunberg, S. M.; Hesketh, P. J. “Control of Chemotherapy-Induced emesis” N. Engl. J. Med. 1993, 329, 1790-96. Ondansetron is indicated for prevention of nausea and vomiting associated with some cancer chemotherapy, radiotherapy and postoperative nausea and/or vomiting.

The hydrochloride salt of ondansetron is generally safe for oral administration to a patient without causing irritation or other adverse effect. The hydrochloride salt is marketed in tablet form and in oral solution form under the brand name Zofran®. The tablet's active ingredient is a dihydrate of ondansetron hydrochloride containing two molecules of bound water in ondansetron hydrochloride's crystal lattice.

The present invention relates to the solid state physical properties of ondansetron hydrochloride. These properties can be influenced by controlling the conditions under which the hydrochloride salt is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid can have therapeutic consequences since it imposes an upper limit on the rate at which an orally-administered active ingredient can reach the patient's bloodstream. The rate of dissolution is also a consideration in formulating syrups, elixirs and other liquid medicaments. The solid state form of a compound may also affect its behavior on compaction and its storage stability.

These important physical characteristics are influenced by the conformation and orientation of molecules in the unit cell, which defines a particular polymorphic form of a substance. Llacer and coworkers have postulated that different spectroscopic characteristics of samples of ondansetron free base prepared differently could be attributable to two different configurations about the methylene bridge between the 1,2,3,9-tetrahydrocarbazol-4-one ring and the imidazole ring. Llacer, J. M.; Gallardo, V.; Parera, A. Ruiz, M. A. Intern.J.Pharm., 177, 1999, 221-229.

A crystalline polymorphic form of a compound may exhibit different thermal behavior from amorphous material or another polymorphic form. Thermal behavior is measured in the laboratory by such techniques as capillary melting point, thermogravimetric analysis (TGA) and differential scanning calorimetry (DSC) and can be used to distinguish some polymorphic forms from others. A particular polymorphic form may also give rise to distinct spectroscopic properties that may be detectable by powder X-ray crystallography, solid state ¹³C NMR spectrometry and infrared spectrometry. There is a wide variety of techniques that have the potential of producing different crystalline forms of a compound. Examples include crystallization, crystal digestion, sublimation and thermal treatment.

U.S. Pat. No. 4,695,578, Example 1a, discloses a preparation of ondansetron by alkylation of 2-methylimidazole with 2,3,4,9 tetrahydro-N,N,N,9-tetramethyl-4-oxo-1H-carbazole-3-methanaminium iodide. In this example, ondansetron was isolated as its hydrochloride salt by suspending the reaction product in a mixture of absolute ethanol and ethanolic HCl, warming the suspension, filtering to remove impurities and precipitating the hydrochloride salt with dry ether.

In Example 10 of the '578 patent, ondansetron free base was converted into a hydrochloride salt dihydrate by dissolving the free base in a mixture of isopropanol and water and treating it with concentrated hydrochloric acid. After filtration at elevated temperature, ondansetron was driven out of solution by adding additional isopropanol and cooling. The dihydrate was obtained as a white crystalline solid by recrystallizing it from a 6:10 mixture of water and isopropanol. Ondansetron hydrochloride dihydrate obtained by following Example 10 of the '578 patent is denominated Form A in this disclosure. Powdered samples of Form A produce a powder X-ray diffraction pattern essentially the same as the pattern shown in FIG. 1.

U.S. Pat. No. 5,344,658 describes ondansetron having a particular particle size distribution and the use of such ondansetron in a pharmaceutical composition. The particle size of ondansetron hydrochloride dihydrate obtained by crystallization from a solvent is reduced by desolvating them, e.g. by heating, and then exposing the desolvated crystals to a humid atmosphere. A collection of crystals obtained by this particle size reduction process is said to consist exclusively of crystals of less than 250 micron size and to contain 80% or more crystals of less than 63 microns. Crytals size was determined by air jet seive analysis.

According to the '658 patent, ondansetron hydrochloride dehydrate having the same particle size distribution as the rehydrated ondansetron hydrochloride also is provided as part of that invention. Since only one process for dehydrating ondansetron hydrochloride is described in the '658 patent, a dehydrate is evidently the intermediate compound that is rehydrated in the particle size reduction process.

U.S. Pat. Nos. 4,695,578 and 5,344,658 are incorporated herein by reference.

The discovery of new polymorphic forms of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic. Six new polymorphic forms and solvates of ondansetron hydrochloride have now been discovered.

### SUMMARY OF THE INVENTION

An objective of the present invention is to provide new forms of ondansetron hydrochloride and processes for preparing them.

Accordingly, the present invention provides a novel ondansetron hydrochloride monohydrate that can be prepared either from an ondansetron hydrochloride dihydrate or from ondansetron free base according to methods of the invention. The monohydrate is referred to as a Form A hydrochloride salt due to the similarity of X-ray spectral characteristics to a known dihydrate of ondansetron hydrochloride.

The invention further provides a new anhydrous ondansetron hydrochloride form that has been demonominated Form B. Form B has advantageous particle size characteristics and it is only slightly hygroscopic. Form B may be prepared from ondansetron hydrochloride Form A and from ondansetron free base.

Additional ondansetron hydrochloride forms denominated Forms C, D and H, and processes for preparing them, are also disclosed.

Yet further, the present invention provides isopropanolates and methanolates of ondansetron hydrochloride and processes for preparing them.

The ondansetron hydrochloride anhydrous forms and hydrates of the present invention are suitable for use in pharmaceutical compositions formulated for prevention of post-operative nausea and nausea incurred during a course of chemotherapy.

### BRIEF DESCRIPTION OF THE DRAWINGS

FIG. 1 is a powder X-ray diffraction pattern of ondansetron hydrochloride Form A.

FIG. 2 is a powder X-ray diffraction pattern of ondansetron hydrochloride Form B.

FIG. 3 is a powder X-ray diffraction pattern of ondansetron hydrochloride Form C.

FIG. 4 is a powder X-ray diffraction pattern of ondansetron hydrochloride Form E.

FIG. 5 is a thermogravimetric analysis profile of ondansetron hydrochloride Form E.

FIG. 6 is a powder X-ray diffraction pattern of ondansetron hydrochloride Form H.

FIG. 7 is a powder X-ray diffraction pattern of ondansetron hydrochloride Form I.

FIG. 8 is a thermogravimetric analysis profile of ondansetron hydrochloride Form I.

### DETAILED DESCRIPTION OF THE INVENTION

Ondansetron Hydrochloride Monohydrate

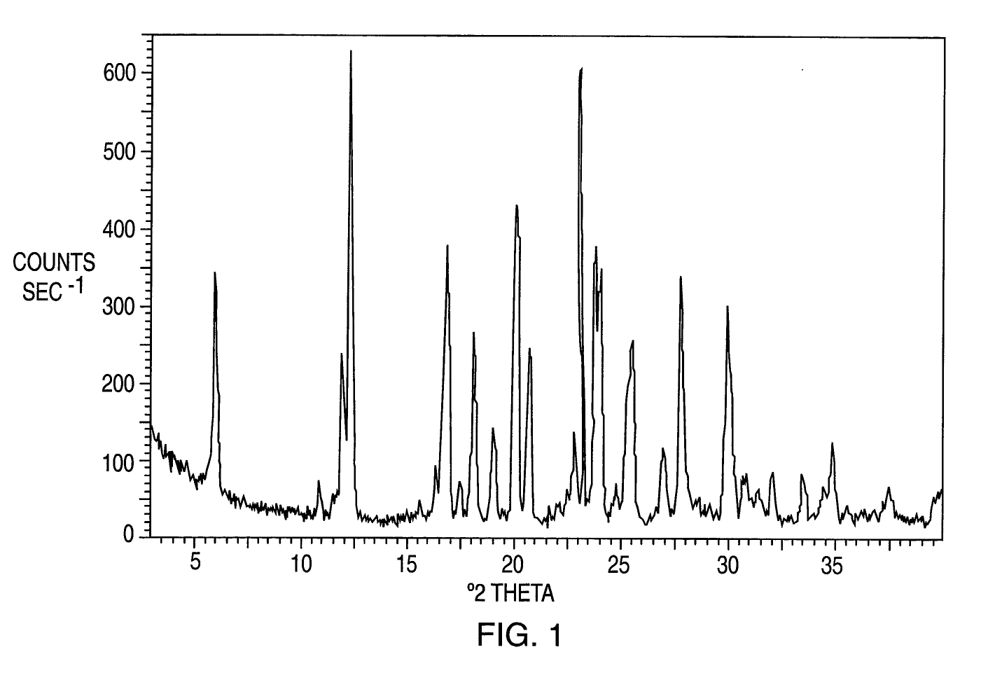

In one aspect, the present invention provides an ondansetron hydrochloride monohydrate. The monohydrate has been found to adopt the same unit cell as the hydrochloride dihydrate obtained by following the procedure of Example 10 of U.S. Pat. No. 4,695,578, which is denominated Form A in this disclosure. Evidence that the monohydrate adopts and/or retains crystalline Form A (depending upon the process by which it is made) is to be found in the X-ray diffraction pattern obtained from the monohydrate, which closely matches the pattern obtained from samples of the Form A dihydrate. This is strong evidence that the crystal structures are approximately the same. Ondansetron hydrochloride Form A is characterized by a strong diffraction at 23.3±0.2 degrees two-theta, and other diffraction peaks at 6.1, 12.4, 17.0, 18.3, 19.2, 20.3, 20.9, 24.1, 25.8, 28.1, 30.3±0.2 degrees two-theta. An X-ray diffraction pattern of a sample of Form A monohydrate is provided as FIG. 1. The ondansetron hydrochloride Form A that one isolates by the methods of this invention are typically large, plate-shaped crystals.

Ondansetron hydrochloride Form A may exist in intermediate degrees of hydration between the monohydrate and dihydrate level. Ondansetron hydrochloride Form A can be crystallized under conditions disclosed herein with varying yet predictable levels of water. The amount of water present in any of the ondansetron hydrate forms of the present invention may be determined by conventional means such as, by the Karl Fisher method.

Exposure of the freshly prepared samples of ondansetron hydrochloride Form A monohydrate to an atmosphere with controlled humidity, such as 60% relative humidity or higher, causes the water level in the crystals to increase rapidly until the dihydrate water content level of about 10.0% is attained. The water uptake usually occurs within a few hours or, at most, overnight. The ease of dehydration of ondansetron hydrochloride Form A dihydrate to a lesser state of hydration and the ability of the lower hydrates to rehydrate under moist atmosphere to the dihydrate level demonstrates that at least one of the waters of crystallization in ondansetron hydrochloride dihydrate is labile.

Upon drying ondansetron hydrochloride Form A dihydrate in a vacuum oven at 90° C. for 12 hours, ondansetron Form A monohydrate may be dehydrated to an essentially anhydrous state having a water content of 1.3% or less. Ondansetron Form A having such a low water content also retains the crystal structure of ondansetron hydrochloride Form A, and therefore is characterized by the powder X-ray diffraction pattern of ondansetron hydrochloride Form A. The highly dehydrated ondansetron hydrochloride Form A rehydrates upon exposure to 50% to 60% relative humidity and is transformed into ondansetron hydrochloride dihydrate (10.0% water).

Preparation of Ondansetron Hydrochloride Form A Monohydrate from Ondansetron Hydrochloride Form A Dihydrate

Ondansetron hydrochloride Form A monohydrate may be prepared from ondansetron hydrochloride Form A dihydrate. The dihydrate is suspended or slurried in a liquid media of aqueous ethanol. Preferred liquid media are mixtures of from about 50% ethanol/water to about 96% ethanol/water. There is not a direct correlation between the hydration level of the Form A obtained and the proportion of water in the liquid medium. Mixtures of water and ethanol falling throughout the range yield Form A with a measured water content consistent with the calculated water content of an ondansetron hydrochloride monohydrate of 5.18 %, as can be seen, for example, by comparison of Examples 14 and 15 below.

The suspension or slurry of the Form A dihydrate is preferably refluxed to accelerate the partial dehydration that occurs in these ethanol and water mixtures. Form A monohydrate may be conveniently separated from the liquid medium by cooling and filtering the suspension.

The process is further illustrated with Examples 12-19. Examples 18 and 19 illustrate that the monohydrate also may be obtained using certain non-aqeuous liquid media, specifically ethanol/isopropanol and ethanol/toluene mixtures. However, such mixtures generally cause ondansetron hydrochloride Form A to crystallize in an intermediate state of hydration between monohydrate and dihydrate, as illustrated with Examples 20-25. Ondansetron hydrochloride having a water content between 6 and 9%, intermediate between the monohydrate (5.18%) and dihydrate (9.85%) is reproducably obtained by following the procedures of Examples 20-25.

Preparation of Ondansetron Hydrochloride Form A from Ondansetron Base

Known processes for making ondansetron hydrochloride Form A have used, as solvent, mixtures of water and isopropanol and water/isopropanol/acetic acid when forming the ondansetron hydrochloride salt from the free base. These solvent systems consistently cause ondansetron hydrochloride to crystallize as the dihydrate.

The present invention provides a new process for making ondansetron hydrochloride Form A from ondansetron free base. In this novel process, the free base is suspended in absolute ethanol and treated with a slight excess of anhydrous HCl. The HCl may be provided either as a gas or dissolved in an organic solvent such as absolute ethanol, toluene, methyl ethyl ketone, isopropanol or ether. The suspension is preferably heated to reflux to hasten the dissolution of the free base and its conversion to the HCl salt. Form A dihydrate is conveniently obtained by cooling the solution to induce crystallization and filtering to separate the solvent and any impurities. The process is further illustrated by Examples 1-11.

We have also found that by using a chlorinated solvent like chloroform, optionally in mixture with water, that we can obtain ondansetron hydrochloride as a monohydrate, as further illustrated in Examples 8-11.

Anhydrous Ondansetron Hydrochloride Form B

The present invention provides a new form of ondansetron hydrochloride designated ondansetron hydrochloride Form B anhydrous and methods for making ondansetron hydrochloride Form B anhydrous. Ondansetron hydrochloride Form B anhydrous can be prepared starting from ondansetron hydrochloride Form A or starting from ondansetron base.

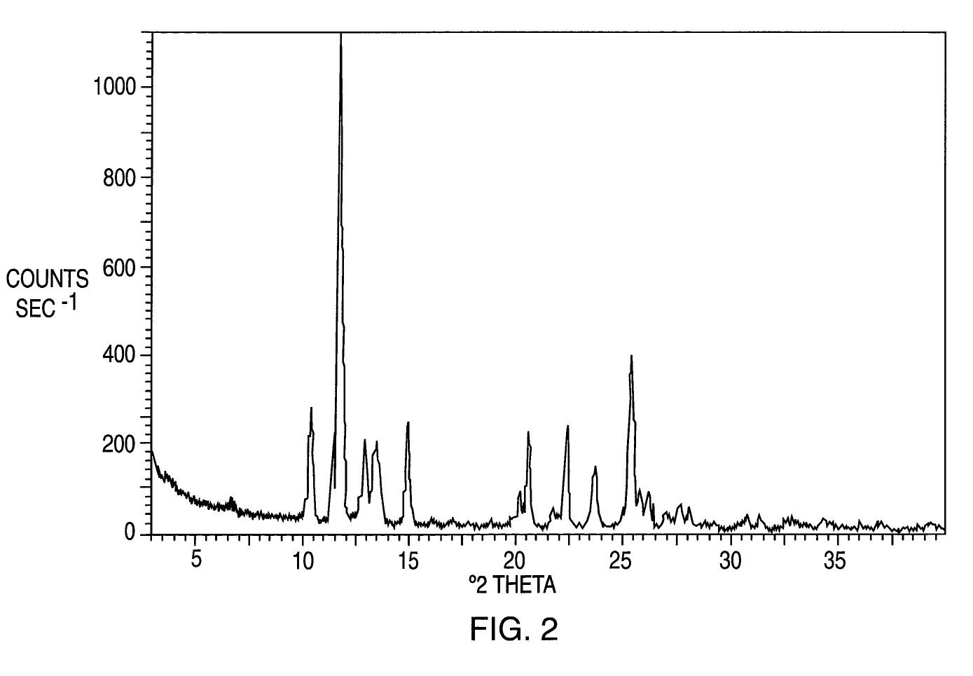

Ondansetron hydrochloride Form B anhydrous is characterized by a strong powder X-ray diffraction peak at 11.9±0.2 degrees two-theta, and powder X-ray diffraction peaks at 10.5, 13.0, 13.5, 15.1, 20.9, 22.7, 24.0, 25.7±0.2 degrees two-theta. An X-ray diffraction pattern of a sample of Form B is provided as FIG. 2. In our hands, anhydrous ondansetron hydrochloride Form B appears as a fine powder composed primarily of small needles and rods.

Ondansetron hydrochloride Form B anhydrous of the present invention absorbs up to 2% moisture when exposed to 60% relative humidity. The water absorbed by the crystal is not within the crystal structure of a hydrous form as a hydrate water. The absence of hydrate water within the crystal structure may be monitored by conventional means, such as, by PXRD. Using X-Ray powder diffraction techniques, the absence of hydrate water is indicated by the absence of ondansetron hydrochloride Form A in the sample. The presence of Form A is indicated by the appearance of a strong peak at 12.3 °2θ in X-ray diffraction pattern of a sample.

The present invention also provides for the preparation of small particles of ondansetron hydrochloride Form B which has the benefit of not requiring expensive and high energy consuming processes, such as, massive milling, or the complex process of dehydrating and rehydrating, in order to achieve the desired particle reduction. The particle size distribution of ondansetron hydrochloride Form B, which is characterized by having small needle/rod shaped particles, with maximum size up to 200 microns, typically with a d(0.9) up to 140 microns, d(0.5) up to 30 microns, d(0.1) up to 2 microns. Preferably, the d(0.9) value is up to 40 microns.

Preparation Anhydrous Ondansetron Hydrochloride Form B from Ondansetron Hydrochloride Form A

By the methods of the present invention, ondansetron hydrochloride Form B anhydrous can be made from ondansetron hydrochloride Form A by treating it with a dry C₁-C₄ alcohol solvent like ethanol, isopropanol and 1-butanol, or a ketone solvent like acetone an methyl ethyl ketone (“MEK”). When the present method for making ondansetron hydrochloride Form B anhydrous is performed at room temperature, the preferred solvent is acetone, methyl ethyl ketone, absolute ethanol or a mixture of isopropanol and ethanol (preferably absolute ethanol is also used in the mixture). As used in this disclosure absolute ethanol refers to ethanol containing no more than 0.5% water. Preferably the isopropanol and ethanol mixture has a 40:65 (v/v) ratio of isopropanol to ethanol. When the present method for making ondansetron hydrochloride Form B anhydrous is performed at elevated temperatures, the preferred solvent is 1-butanol and the mixture is heated to reflux.

The method of the present invention provides the surprising result that ondansetron hydrochloride Form A may be transformed into anhydrous ondansetron hydrochloride Form B by slurrying ondansetron hydrochloride Form A in absolute ethanol, preferably at room temperature (that is, about 20° C.), facilitates a simple and quick transformation of ondansetron hydrochloride Form A to anhydrous ondansetron hydrochloride Form B. The transformation of ondansetron hydrochloride Form A to ondansetron hydrochloride Form B anhydrous is completed between a few hours and up to two days or more, depending upon different parameters like particle size, the relative amount of the solvent, temperature. Typically, complete conversion requires between 24 and 48 hours at room temperature. The reaction should be peformed under dry conditions. Performing the reaction either under a dry nitrogen or argon atmosphere or in a flask that communicates with air through a drying tube containing CaCl₂ provides sufficiently dry conditions.

Ondansetron hydrochloride Form B anhydrous can also be prepared by bubbling HCl gas through a solution of ondansetron base in refluxing toluene.

Preparation of Ondansetron Hydrochloride Form B Anhydrous from Ondansetron Base

The present invention also provides a method for making ondansetron hydrochloride Form B anhydrous from ondansetron free base. By the present methods, ondansetron base is reacted with dry HCl in dry organic solvent. The HCl may be provided either as a gas or dissolved in a dry organic solvent such as absolute ethanol, toluene, methyl ethyl ketone, isopropanol or ether. Upon completion of the reaction, ondansetron hydrochloride Form B anhydrous may be isolated by filtration. Form B crystals have a characteristic needle-shape.

Preparation of ondansetron hydrochloride Form B anhydrous by the present procedure is enabled by the fact that the solvent (ethanol) and the HCl/ethanol solution are dry. Thus, by this way Form A is not formed during the reaction. The reaction can be performed at room temperature (rt) or at reflux. At room temperature, the reaction is heterogeneous and results in ondansetron hydrochloride Form B anhydrous with small particle size distribution. When performed at reflux, the reaction is homogenous, and it can be thus be treated with activated carbon to obtain a purer salt. After hot filtration to remove the carbon, ondansetron hydrochloride Form B may be obtained by cooling the filtrate to room temperature and recovering precipitated Form B by filtration. The particle size distribution can be easily controlled by varying the crystallization parameters, including by controlled cooling.

Ondansetron Hydrochloride Form C

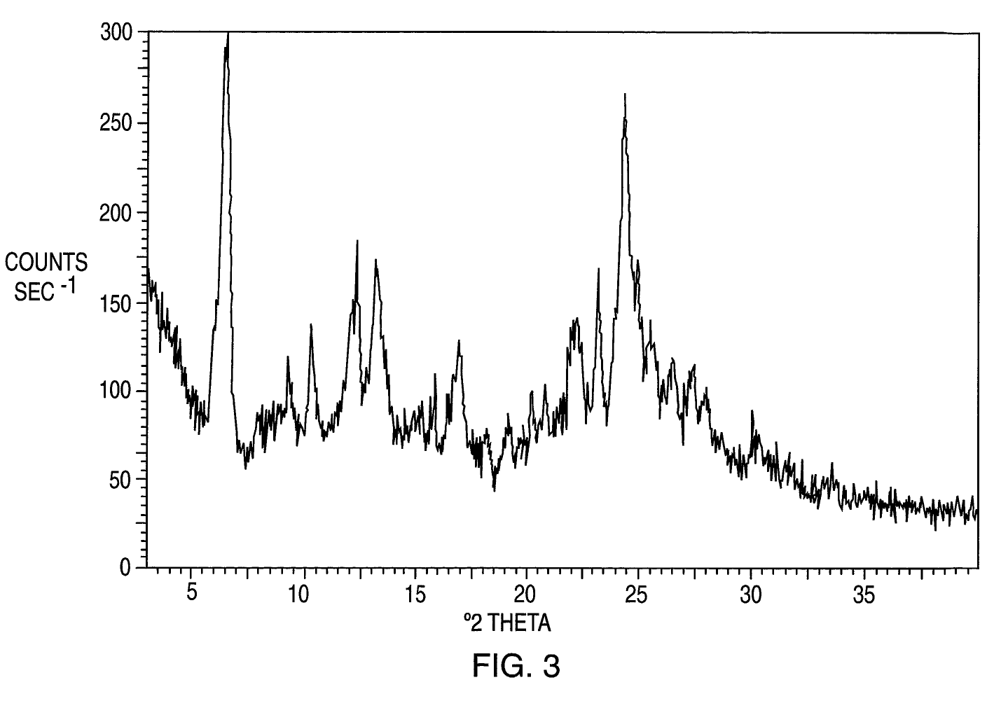

The present invention provides a new form of ondansetron hydrochloride designated ondansetron hydrochloride Form C and methods for making ondansetron hydrochloride Form C. This form is characterized by strong powder X-ray diffraction peaks at 6.3, 24.4, degrees two-theta and other typical peaks at 9.2, 10.2, 13.1, 16.9 degrees two-theta. An X-ray diffraction pattern of a sample of Form C is provided as FIG. 3. This form may be obtained by dissolving ondansetron hydrochloride Form A in ethanol at reflux after addition of HCl (gas or in solution). After cooling the solution, the precipitate is filtered and the mother liquor is evaporated under reduced pressure. Ondansetron hydrochloride Form C results from this solid obtained after evaporation. Ondansetron hydrochloride Form C is hygroscopic and may contain up to 10% water.

Ondansetron Hydrochloride Form D

The present invention provides a new form of ondansetron hydrochloride designated ondansetron hydrochloride Form D. This form may be obtained as a mixture with ondansetron hydrochloride Form C. Ondansetron hydrochloride Form D is obtained by dispersing ondansetron hydrochloride Form A in about 1 milliliter of xylene per gram of Form A, then melting the dispersion at a temperature above 150° C., preferably above 180° C., and pouring the melt into cold alcohols, preferably about 10 milliliters of ethanol per gram of the dispersion. The alcohol can be at a temperature below room temperature up to room temperature, preferably at about −10° C.

Ondansetron hydrochloride Form D is characterized by powder X-ray diffraction peaks at 8.3, 14.0, 14.8, 25.5 degrees two-theta.

Ondansetron Hydrochloride Form E

The present invention provides a new form of ondansetron hydrochloride designated ondansetron hydrochloride Form E and methods for making ondansetron hydrochloride Form E.

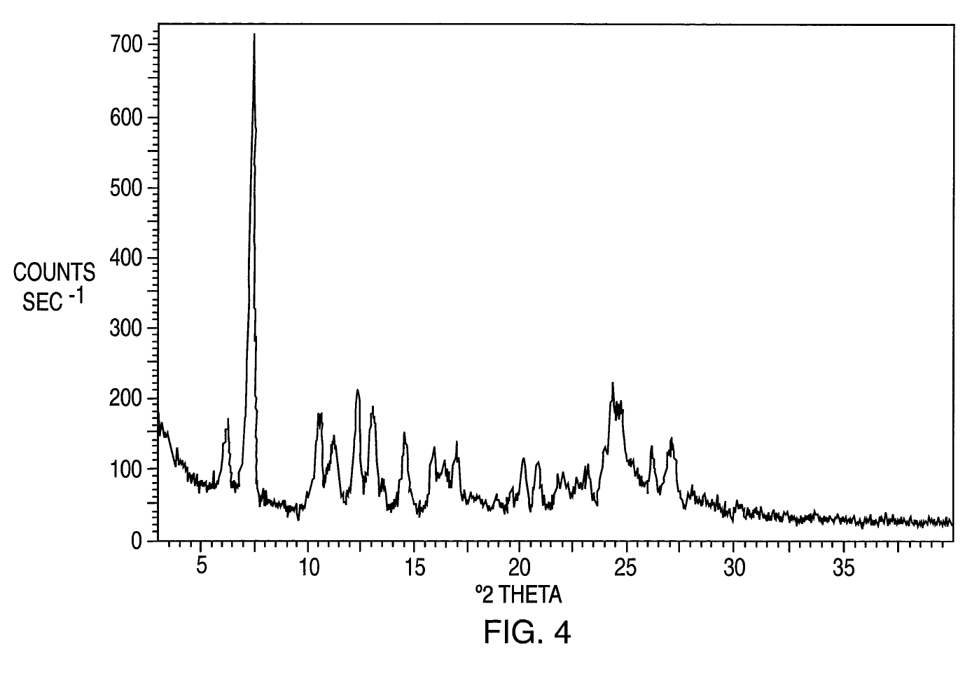

Ondansetron hydrochloride Form E is characterized by a strong powder X-ray diffraction peak at 7.4 degrees two-theta and other typical peaks at 6.3, 10.5, 11.2, 12.3, 13.0, 14.5, 15.9, 17.0, 20.1, 20.8, 24.5, 26.2, 27.2 degrees two-theta. An X-ray diffraction pattern of a sample of Form E is provided as FIG. 4. Ondansetron hydrochloride Form E contains 1.8%-2.0% water, as measured by Karl Fisher. This is a stoichiometric value corresponding to ⅓ molecule of water per molecule of ondansetron hydrochloride (theoretical value: 1.8%).

It was surprisingly found that treating ondansetron hydrochloride Form A in isopropanol results in the formation of ondansetron hydrochloride Form E.

Ondansetron hydrochloride, preferably the Form A dihydrate, can be treated in isopropanol at room temperature or at reflux temperature, to yield ondansetron hydrochloride Form E.

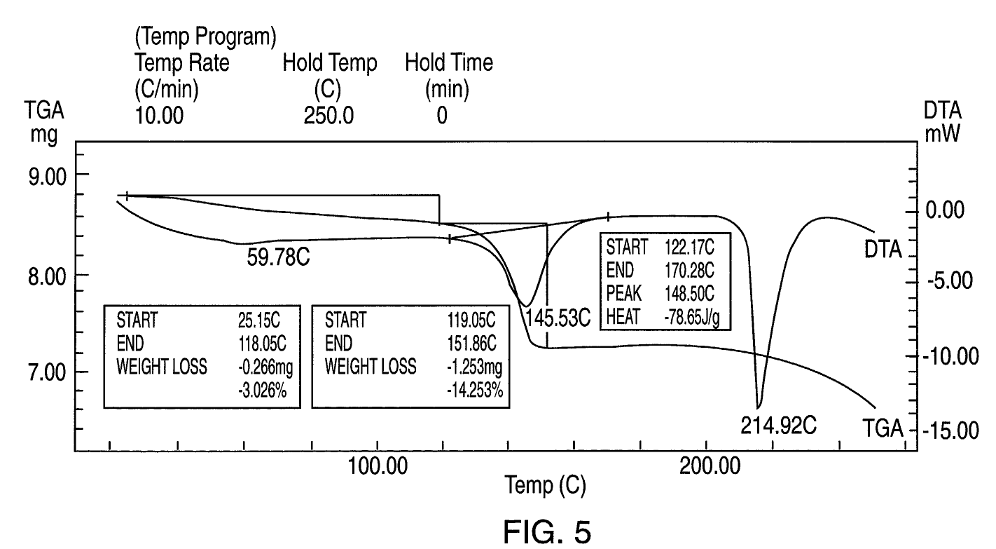

It was found that ondansetron hydrochloride Form E, which is obtained by treating ondansetron hydrochloride Form A in isopropanol, includes quantities of isopropanol of about 8-10% or 14%. A typical TGA curve of ondansetron hydrochloride Form E (FIG. 5) shows a weight loss of about 2% up to about 120° C., and a sharp weight loss at about 150° C. of 9% or 14%. According to stoichiometric computations, ondansetron hydrochloride Form E can exist as a monosolvate of isopropanol or a hemisolvate of isopropanol (the expected stoichiometric value of isopropanol hemisolvate is 8.4%, and the expected stoichiometric value of isopropanol monosolvate is 15.4%). It was also found that ondansetron hydrochloride propanolate Form E when exposed up to 60% relative humidity for one week can contain water up to 10% without modifying its crystal structure.

Ondansetron Hydrochloride Form H

The present invention provides a new form of ondansetron hydrochloride designated ondansetron hydrochloride Form H and methods for making ondansetron hydrochloride Form H. By the methods of the present invention, ondansetron hydrochloride Form H is obtained by dissolving ondansetron base in ethanol, preferably absolute ethanol, adding an amount of an ethanol/hydrochloric acid solution sufficient to provide 1.5 equivalents of HCl, and precipitating ondansetron hydrochloride Form H by adding t-butyl methy ether or diethyl ether (preferably dry and freshly distilled) to facilitate precipitation (1 g/86 ml). The solution of ondansetron base in absolute ethanol may be heated above room temperature, preferably at about 45° C. Ondansetron hydrochloride Form H may also be obtained in a mixture with ondansetron hydrochloride Form B anhydrous when ethyl ether is used as the solvent. Ondansetron hydrochloride Form H isolated contained about 2% water content.

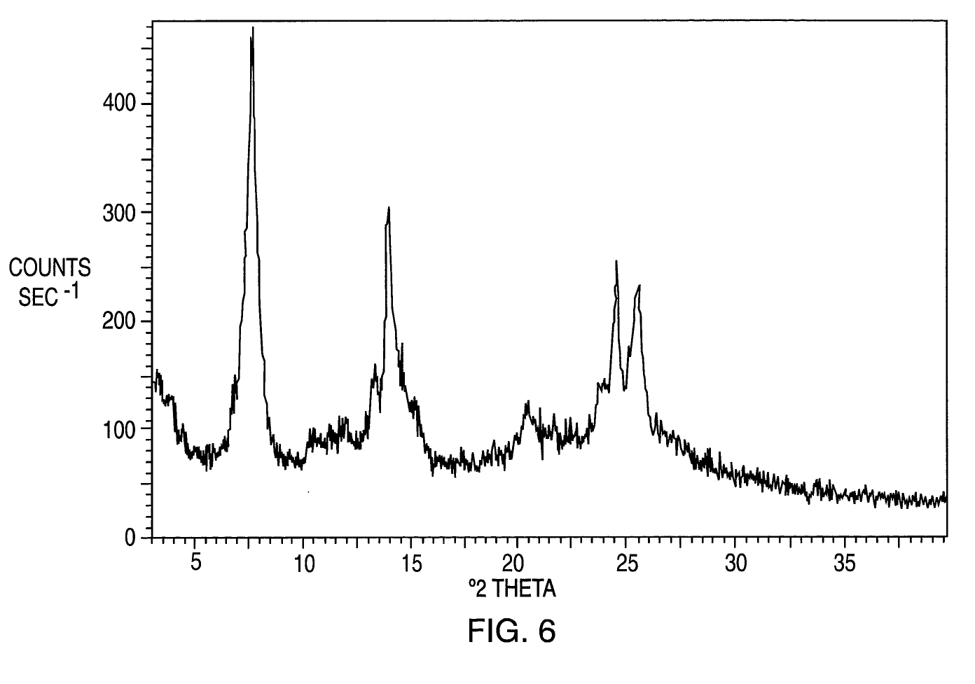

Ondansetron hydrochloride Form H is characterized by unique powder X-ray diffraction peaks at 7.8, 14.0, 14.8, 24.7, 25.6 degrees two-theta. An X-ray diffraction pattern of a sample of Form H is provided as FIG. 6.

Ondansetron Hydrochloride Form I

The present invention provides a new form of ondansetron hydrochloride designated ondansetron hydrochloride Form I and methods for making ondansetron hydrochloride Form I. Ondansetron hydrochloride, either Form A or anhydrous, can be treated in methanol vapors for a period of few days to two weeks, to yield ondansetron hydrochloride Form I. In order to obtain conversion of most of the sample to Form I, a period of two weeks is needed. Ondansetron hydrochloride Form I contains 3.1% water, as measured by Karl Fisher. This is a stoichiometric value correspondent to ½ molecule of water per molecule of ondansetron hydrochloride (theoretical value: 2.5%). Ondansetron hydrochloride Form I contains methanol up to 10% which roughly corresponds to the monomethanolate stoichiometric value of about 9%.

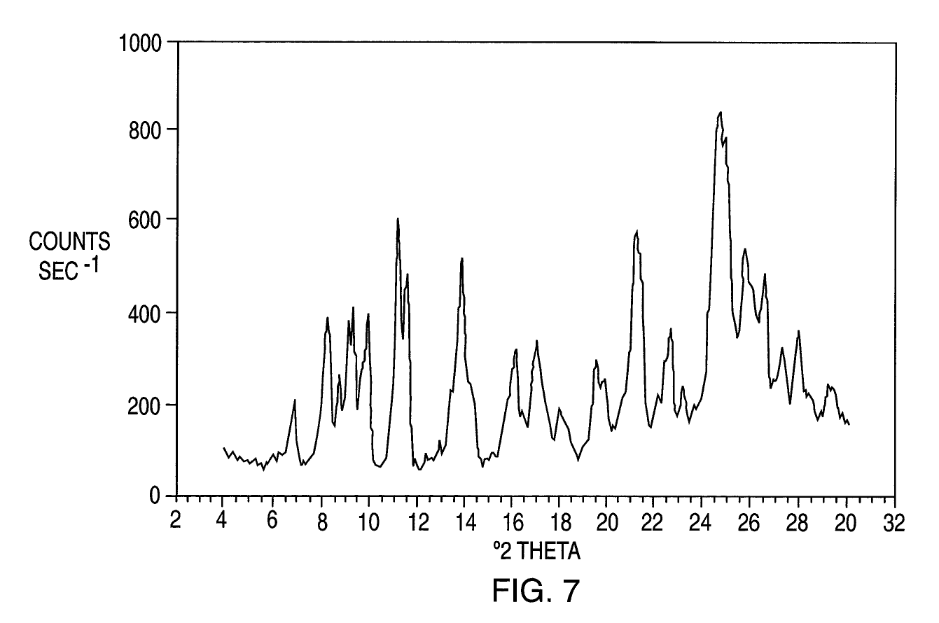

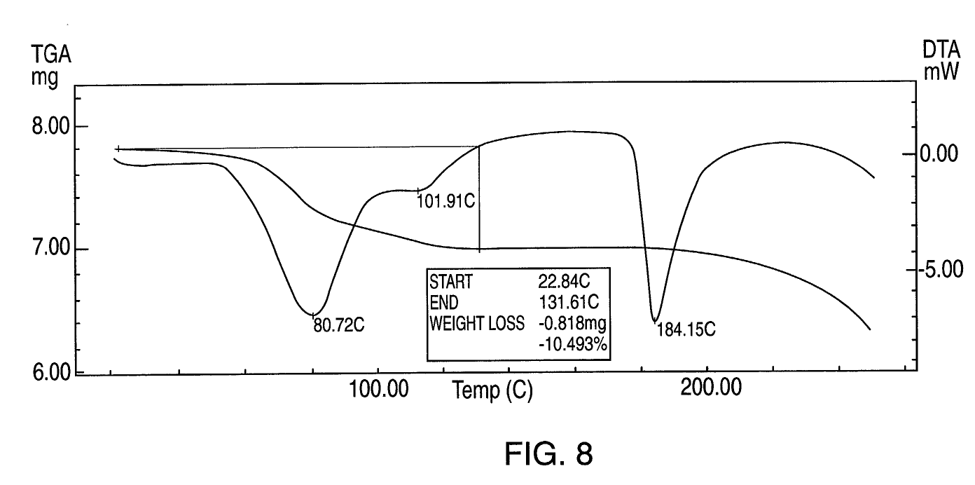

Ondansetron hydrochloride Form I is characterized by a strong XRD peak at 24.9 degrees two-theta and other XRD peaks at 6.9, 8.2, 8.7, 9.1, 9.3, 9.9, 11.1, 11.6, 13.8, 16.1, 16.9, 17.9, 21.1, 22.7, 25.7, 26.6, 27.4, 27.9±0.2 degrees two-theta. An X-ray diffraction pattern of a sample of Form I is provided as FIG. 7. A typical thermogravimetric analysis curve of Form I (FIG. 8) shows a weight loss of about 10% in the range of room temperature to about 130° C.

In accordance with the present invention, the present new forms of ondansetron hydrochloride may be prepared as pharmaceutical compositions that are particularly useful in the treatment of a variety of conditions, including the prevention of nausea and vomiting associated some cancer chemotherapy, radiotherapy and postoperative nausea and/or vomiting. Such compositions comprise one of the new forms of ondansetron hydrochloride with pharmaceutically acceptable carriers and/or excipients known to one of skill in the art.

Preferably, these compositions are prepared as medicaments to be administered orally, or intravenously. Suitable forms for oral administration include tablets, compressed or coated pills, dragees, sachets, hard or gelatin capsules, sub-lingual tablets, syrups and suspensions. While one of ordinary skill in the art will understand that dosages will vary according to the indication, age of the patient, etc., generally polymorphic and hydrate forms of ondansetron hydrochloride of the present invention will be administered at a daily dosage of about 8 to about 32 mg per day, and preferably about 8 to about 24 mg per day, and preferably about 8 to about 24 mg per day. Additionally, new forms of ondansetron hydrochloride of the present invention may be administered as a pharmaceutical formulation comprises new forms of ondansetron hydrochloride in an amount of about 4 mg to about 32 mg per tablet. Preferably, the new forms of ondansetron hydrochloride of the present invention may be administered as a pharmaceutical formulation comprises new forms of ondansetron hydrochloride in an amount of 4 mg, 8 mg, or 24mg per tablet. Additionally, the new forms of ondansetron hydrochloride of the present invention may be administered as an oral solution comprises new forms of ondansetron hydrochloride in an amount 4 mg of ondansetron per 5 ml.

### EXAMPLES

The powder X-ray diffraction patterns were obtained by methods known in the art using a Philips powder X-ray diffractometer, Phillips Generator TW1830, Goniometer model PW3020, MPD Control PW3710, X-Ray tube with Cu target anode, Monochromator proportions counter, at a scanning speed of of 2° per minute.

The particle size distributions were obtained by methods known in the art by laser diffraction technique; using a Malvern Laser Diffraction Mastersizer S, equipped with a small volume cell of 50-80 ml as the flow cell. The samples was dispersed using silicon fluid F-10 as the diluent and by adding a small aliquot of sample in 5 ml diluent inside a 10 ml glass bottle. The suspension was mixed by vortex 5 seconds, and then sonicated in the open bottle for 2 and a half minutes to break hard aggregates. The suspension was added dropwise in the flow cell filled with diluent until the required obscuration (15-28%) was achieved. The measurement was started after one minute recirculation at about 1700-1800 rpm pump speed.

As known in the art, the experimental conditions like sonication, vortex or any other dispersion medium are meant to disperse the partilces and break aggregates that may be present in the material as a result of sticking of particles during drying for instance, with the purpose to provide an accurate particle size distribution of primary particles. Hence, the experimental conditions used may vary according to the appearance of the samples, and the presence of aggregates.

Preparation of Ondansetron Form A with Different Levels of Hydration from Ondansetron Free Base

Example 1: Ondansetron base (400 mg, 1.36×10⁻³ mole) was suspended in 40 ml of absolute ethanol at room temperature. The suspension was heated to reflux to dissolve the ondansetron. After 20 min. of stirring at reflux, an ethanolic solution containing 1.1 equivalents of HCl was added. The reaction mixture was stirred at this temperature for an additional 10 min and then cooled slowly to 0° C. After stirring at 0° C. for 1 hour, the solid was filtered under vacuum and dried under vacuum at 50° C. to give 90 mg of ondansetron hydrochloride Form A. KF=10%.

Example 2: Ondansetron base (400 mg, 1.36×10⁻³ mole) was suspended in 12 ml of absolute ethanol at room temperature. The suspension was heated to reflux to dissolve the ondansetron. After 20 min. of stirring at reflux, an ethanolic solution containing 1.1 equivalents of HCl was added. The reaction mixture was stirred at this temperature for an additional 10 min and then cooled slowly to 0° C. After stirring at 0° C. for 1 hour, the solid was filtered under vacuum and dried under vacuum at 50° C. to give 536 mg of ondansetron hydrochloride Form A. KF=8.1%.

Example 3: Ondansetron base (400 mg, 1.36×10⁻³ mole) was suspended in 16 ml of a 1:1 mixture of ethanol and isopropanol at room temperature. The suspension was heated to reflux to dissolve the ondansetron. After 20 min. of stirring at reflux, an ethanolic solution containing 1.1 equivalents of HCl was added. The reaction mixture was stirred at this temperature for an additional 10 min. Evaporation of the solvent gave ondansetron hydrochloride dihydrate Form A.

Example 4: Ondansetron base (400 mg, 1.36×10⁻³ mole) was suspended in 40 ml of absolute ethanol at room temperature. The suspension was heated to reflux to dissolve the ondansetron. After 20 min. of stirring at reflux, an ethanolic solution containing 1.5 equivalents of HCl was added. The reaction mixture was stirred at this temperature for an additional 10 min and then cooled slowly to 0° C. After stirring at 0° C. for 1 hour, the solid was filtered under vacuum and dried under vacuum at 50° C. to give 320 mg of ondansetron hydrochloride Form A. KF=8.1%.

Example 5: Ondansetron base (400 mg, 1.36×10⁻³ mole) was suspended in 14 ml of absolute ethanol at room temperature. The suspension was heated to reflux to dissolve the ondansetron. After 20 min. of stirring at reflux, an ethanolic solution containing 1.5 equivalents of HCl was added. The reaction mixture was stirred at this temperature for an additional 10 min. Evaporation of the solvent gave 280 mg ondansetron hydrochloride Form A. KF=9.3%.

Example 6: Ondansetron base (400 mg, 1.36×10⁻³ mole) was suspended in 12 ml of absolute ethanol at room temperature. Four angstrom molecular sieves were added to the flask. The suspension was then heated to reflux to dissolve the ondansetron. After 20 min. of stirring at reflux, an ethanolic solution containing 1.5 equivalents of HCl was added. The reaction mixture was stirred at this temperature for an additional 10 min and then cooled slowly to 0° C. After stirring at 0° C. for 1 hour, the solid was filtered under vacuum and dried under vacuum at 50° C. to give 296 mg of ondansetron hydrochloride Form A. KF=9.5%.

Example 7: Ondansetron base (400 mg, 1.36×10⁻³ mole) was suspended in 20 ml of absolute ethanol at room temperature. The suspension was heated to reflux to dissolve the ondansetron. After 20 min. of stirring at reflux, a solution containing 1.1 equivalents of HCl in isopropanol was added. The reaction mixture was stirred at this temperature for an additional 10 min and then cooled slowly to 0° C. After stirring at 0° C. for 1 hour, the solid was filtered under vacuum and dried under vacuum at 50° C. to give 290 mg of ondansetron hydrochloride Form A. KF=9.5%.

Example 8: Ondansetron base (2.5 g, 8.5×10⁻³ mole) was dissolved in 80 ml of chloroform at room temperature. Then 1.1 eq of HCl gas was bubbled into the solution over 20 min. The reaction mixture was stirred at room temperature for an additional 30 min. The solid was filtered under vacuum and dried under vacuum at 50° C. to give 2.8 g of ondansetron hydrochloride Form A. KF=5.4%.

Example 9: Ondansetron base (2.5 g, 8.5×10⁻³ mole) was dissolved in 87.5 ml of chloroform at room temperature. Then 1.1 eq of HCl gas was bubbled into the solution over 20 min. The reaction mixture was stirred at room temperature for an additional 30 min. The solid was filtered under vacuum and dried under vacuum at 50° C. to give 2.5 g of ondansetron hydrochloride Form A.

Example 10: Ondansetron base g (5 g, 17.06×10⁻³ mole) was dissolved in 175 ml of chloroform at room temperature. Then HCl gas was bubbled at room temperature for 15 min. 0.6 equivalent of H₂O was added slowly to the reaction mixture. The reaction mixture was stirred at room temperature for an additional 3 hrs. Then, the solid was filtered under vacuum and dried under vacuum at 50° C. to give 6.3 g of ondansetron hydrochloride Form A. KF=8.4%.

Example 11: Ondansetron base (5 g, 17.06×10⁻³ mole) was suspended in a mixture of H₂O/CHCl₃ (140/20 v/v) at room temperature. The reaction mixture was heated to reflux temperature and then 1.1 eq. of 1 N aqueous HCl was added by syringe pump at 1 ml/min. The reaction mixture was stirred at room temperature for 30 min. and then slowly cooled to 5° C. The partial precipitation that was obtained during cooling was filtered (1.7 g) under vacuum and dried under vacuum at 50° C. to give a white solid. The mother liquor was left overnight at room temperature to give an extra precipitate (1.7 g) that was filtered and dried under vacuum. Both fractions gave ondansetron hydrochloride Form A.

Preparation of Ondansetron Form A Monohydrate from Ondansetron Hydrochloride Form A Dihydrate

Example 12: Ondansetron hydrochloride Form A dihydrate (5 g) in 70 ml of a 96% aqueous solution of EtOH was heated to reflux temperature for 22 hrs. The reaction mixture was then allowed to cool to room temperature and then was cooled to 0° C. The solid that precipitated was filtered and dried at 65° C. for 20 hrs, yielding 1.2 g of ondansetron hydrochloride Form A monohydrate, KF =5.4%.

Example 13: Ondansetron hydrochloride Form A dihydrate (5.0 g) in 70 ml of a 90% aqueous solution of EtOH was heated to reflux temperature for 22 hrs. The reaction mixture was allowed to cool to room temperature and then cooled to 0° C. The solid was then filtered, dried at 65° C. for 20 hrs. to give 4.0 g of ondansetron hydrochloride Form A monohydrate; KF =5.0%.

Example 14: Ondansetron hydrochloride Form A dihydrate (5.0 g) was slurried in 70 ml of a 90% aqueous solution of EtOH at room temperature for 22 hrs. The solid was then filtered, dried at 65° C. for 20 hrs. to give 3.5 g of ondansetron hydrochloride Form A monohydrate; KF =5.2%.

Example 15: Ondansetron hydrochloride Form A dihydrate (5 g) was slurried in 70 ml of a 50% aqueous solution of EtOH at room temperature for 22 hrs. Methyl ethyl ketone (100 ml) was then added to preciptate the ondansetron hydrochloride. The mixture was cooled to 0° C. and the precipitate was filtered and dried at 65° C. for 20 hrs. to give 0.4 g of ondansetron hydrochloride Form A monohydrate; KF =5.2%.

Example 16: Ondansetron hydrochloride Form A dihydrate (5 g) was slurried in 70 ml of a 50% aqueous solution of EtOH at room temperature for 22 hrs. The solid was then filtered, dried at 65° C. for 20 hrs. to give 0.4 g of ondansetron hydrochloride Form A monohydrate; KF =5.7%.

Some of the compound was recovered from the mother liquor by adding 125 ml of MEK for precipitation and filtering under vacuum. The solid was dried at 65° C. for 20 hrs. to give 1.7 g of ondansetron hydrochloride Form A monohydrate; KF 5.4%.

Example 17: Ondansetron hydrochloride Form A dihydrate (5 g) was slurried in 70 ml of a 96% aqueous solution of EtOH at room temperature for 22 hrs. The solid was then filtered and dried at 65° C. for 20 hrs. to give 3.8 g of ondansetron hydrochloride Form A; KF =6.1 %.

Example 18: A slurry of 5 g of ondansetron hydrochloride Form A dihydrate in a mixture of EtOH/IPA (40 ml/65 ml) was sonicated for 2 min, amplitude 50%, energy 3.5KJ. Then, the white solid was filtered using a 8 mm filter paper and dried at 65° C. for 20 hrs. to give 2.7 g of ondansetron hydrochloride Form A; KF =4.8%.

Example 19: A 250 ml flask was charged with a suspension of ondansetron hydrochloride Form A dihydrate (5 g) in a mixture of EtOH/Toluene (110 ml/50 ml). The flask was equipped with a distillation apparatus. Forty five milliliters of solvent was distilled off at atmospheric pressure until a clear solution was obtained. The reaction mixture was then allowed to cool to 10° C. over 1 hour. The precipitate was filtered under vacuum and dried in a vacuum oven at 50° C. for 16 hrs. to give 3.7 g of ondansetron hydrochloride Form A; KF =6.1 %.

Preparation of Ondansetron Hydrochloride Form A with a Water Content of Between 6 and 9 Percent.

Example 20: A slurry of 5 g of ondansetron hydrochloride Form A dihydrate in 90% aqueous EtOH (70 ml) was sonicated for 2 minutes with an amplitude of 50%, and an energy 3.5KJ. Then, the white solid was filtered using a 8 micron pore size filter paper and dried at 65° C. for 20 hrs to give 2.7 g of ondansetron hydrochloride Form A; KF =6.6%.

Example 21: A slurry of 5 g of ondansetron hydrochloride Form A dihydrate in a mixture of EtOH/IPA (65 ml/40 ml) was sonicated for 2 min., amplitude 50%, energy 3.5KJ. Then, the white solid was filtered using a 8 micron pore size filter paper and dried at 65° C. for 20 hrs to give 3.6 g of ondansetron hydrochloride Form A; KF =6.7%

Example 22: A slurry of 5 g of ondansetron hydrochloride Form A dihydrate in toluene (100 ml) was heated to 100° C. for 17 hours. The reaction mixture was then cooled to 0° C. The white solid was filtered under vacuum and dried in a vacuum oven at 50° C. for 16 hrs. to give 4.0 g of ondansetron hydrochloride Form A; KF =7.8%.

Example 23: Ondansetron hydrochloride Form A dihydrate (5 g) in absolute EtOH/toluene (45 ml/20 ml) was heated to reflux temperature for a few hours. After stirring at room temperature overnight, the solid was filtered under vacuum and dried in a vacuum oven at 50° C. for 16 hours to give 4.0 g of ondansetron hydrochloride Form A; KF =7.8%.

Example 24: Ondansetron hydrochloride dihydrate Form A (2.1 g) in a mixture of EtOH/toluene (45 ml/20 ml) were heated to reflux temperature. Then 25 ml of the solvent was distilled off at atmospheric pressure. The reaction mixture was then allowed to cool to 10° C. over 3 hrs. The white precipitate was filtered under vacuum and dried in a vacuum oven at 50° C. for 5 hrs. to give 1.4 g of ondansetron hydrochloride Form A; KF =8.8%

Example 25: A slurry of 5 g of ondansetron hydrochloride Form A dihydrate in absolute EtOH (70 ml) was sonicated for 2 minutes with an amplitude of 50% and an energy of 3.5KJ. Then, the white solid was filtered using an 3 micron pore size filter paper and dried at 65° C. for 20 hrs. to give 3.3 g of ondansetron hydrochloride Form A; KF =9.3%

Preparation of Anhydrous Ondansetron Hydrochloride Form B

Example 26: To a flask equipped with a CaCl₂ drying tube 5.0 g of ondansetron HCl Form A and a mixture of IPA/EtOH (40/65 ml) were added. The mixture was stirred at room temperature for 22 hrs. After filtration the obtained solid was dried at 65° C. for 20 hours to give 4.0 g of Ondansetron hydrochloride Form B anhydrous, KF=0.6%.

Example 27: To a flask equipped with a CaCl₂ drying tube, 5.0 g of ondansetron HCl Form A and absolute EtOH (70 ml) were added. The mixture was stirred at room temperature for 22 hrs. After filtration the obtained solid was dried at 65° C. for 20 hrs to give 3.7 g of ondansetron Form B, HCl, KF =0.4%.

Example 28: To a three-necked flask equipped with a condenser, a themometer and a CaCI₂ tube ondansetron base (2.0 g) and 280 ml of toluene were added. The mixture was heated to reflux until a clear solution was obtained. HCl gas was bubbled in until a pH of 1 was achieved. The reaction mixture was refluxed for an additional 1 hour, then cooled to room temperature. The obtained precipitation was filtered and dried at 65° C. for 20 hrs to give 1.7 g of ondansetron Form B HCl, KF=1.6%.

Example 29: Ondansetron base (2.0 g, 6.8×10⁻³ mole) was suspended in MEK (220 ml) for 30 minute until a complete dissolution occurred. Then HCl gas was bubbled until the solution reached pH=1. The reaction mixture was refluxed for an additional 1 hour, cooled to at room temperature, filtered under vacuum and dried at 65° C. for 20 hrs. The white solid obtained was slurried in absolute ethanol (70 ml) at room temperature for 22 hours, using CaCl₂ tube. The reaction mixture was then filtered under vacuum and dried at 65° C. for 20 hrs to give 1.9 g of ondansetron hydrochloride Form B anhydrous.

Example 30: Ondansetron base (3 g) (10.×2 10⁻³ mole) was suspended in MEK (330 ml) for 15 minutes until a complete dissolution occurred. Then an ethanolic solution of HCl (1.5 eq) was added. The reaction mixture was refluxed for an additional 30 minutes, cooled to at room temperature, filtered under vacuum and dried at 65° C. for 20 hrs. The white solid obtained was then slurred in 105 ml of a mixture EtOH abs/IPA (65/40 ml) at room temperature for 22 hours, using CaCl₂ tube. Then the reaction mixture was filtered under vacuum and dried at 65° C. for 20 hrs to give 3.16 g of ondansetron hydrochloride Form B anhydrous.

Example 31: Ondansetron base (5 g) (17.0×10⁻³ mole) was suspended in 250 ml of absolute ethanol, EtOH. Then, an ethanolic solution of HCl (1.5 eq) was added. The reaction mixture was warmed (45° C.) to get a clear solution. The reaction mixture was allowed to cool to room temperature and then dry ether was added (430 ml) to precipitate a solid. The precipitate was filtered under vacuum and dried in oven 65° C. for 24 hours to give 3.16 g of ondansetron hydrochloride Form B anhydrous. KF=1.7%.

Example 32: Ondansetron base (5 g) (17.0×10⁻³ mole) was suspended in 250 ml of absolute ethanol. Then, an ethanolic solution of HCl (1.5 eq) was added. The ethanolic solution was prepared by bubbling HCl gas into absolute ethanol under dry conditions. The reaction mixture was warmed (45° C.) to get a clear solution, and a hot filtration of the clear solution was done. To this filtrate was added, at room temperature, dry ether (430 ml) to precipitate a solid. The precipitate was filtered under vacuum and dried in oven 65° C. for 18 hours to give 3.16 g of ondansetron hydrochloride Form B anhydrous. KF=1.0%

Preparation of Ondansetron Hydrochloride Form C

Example 33: Ondansetron base (1.5 g, 5.11×10⁻³ mole) was dissolved in absolute ethanol(150 ml) freshly distilled at reflux temperature. Then an ethanolic solution of HCl (1.1 eq) was added at reflux. The reaction mixture was stirred for 20 minutes and allowed to cool slowly to room temperature. A very thick precipitate appeared at room temperature. The mixture was then filtered under vacuum to give 536 mg of a white solid. The ethanolic phase was evaporated under reduced pressure to give 824 mg of ondansetron hydrochloride Form C. KF=9.9%

Example 34: Ondansetron base (5 g) (17.0×10⁻³ mole) was suspended in absolute ethanol (150 ml) freshly distilled with 10 g of 4A molecular sieves. The reaction mixture was heated to 80° C. until the complete dissolution of the starting material. Then an ethanolic solution of HCl (1.5 eq) was added dropwise at this temperature and the reaction mixture was stirred for 15 minutes. The mixture was allowed to cool slowly to room temperature and then to 0° C. to complete the precipitation. The solid mixture was then filtered under vacuum, washed 3 times with IPA (3×10 ml) to give 3.07 g of a white solid. The ethanolic phase was left at 4° C. overnight and then the precipitate was filtered under reduced pressure to give 600 mg of a solid. The mother liquor of this fraction was then evaporated under reduced pressure to give 1 g of ondansetron hydrochloride Form C. KF=9.9%

Preparation of Ondansetron Hydrochloride Form D

Example 35: Ondansetron hydrochloride Form A was suspended (5 g) (17.0×10⁻³ mole) in xylene (5 ml). The suspension was heated to above 180° C. until the ondansetron hydrochloride melted. Then the melt was poured slowly into a solution of absolute EtOH (50 ml) at −10° C. The resulting solid was stirred in absolute EtOH for 30 minutes at −10° C. and then gravity filtered. The solid was dried in oven at 65° C. for 18 hours to afford 1.31 g of ondansetron hydrochloride Form D. KF=3.84%

Preparation of Ondansetron Hydrochloride Form E

Example 36: Ondansetron hydrochloride Form A (5 g, 13.6×10⁻³ mole) was slurried in IPA (70 ml), at room temperature overnight. The white solid was then filtered under vacuum and dried in an oven 65° C. for 24 hours to afford 4.9 g of ondansetron hydrochloride Form E as a white solid. KF=1.8%.

Example 37: Ondansetron hydrochloride Form A (5 g, 13.6×10⁻³ mole) was slurried in IPA (40 ml) at reflux temperature overnight. The white solid was filtered under vacuum and dried in oven at 65° C. for 24 hours to afford 5 g of ondansetron hydrochloride Form E as a white solid. KF=2.1%.

Preparation of Ondansetron Hydrochloride Form H

Example 38: Ondansetron base (5 g) (17.0×10⁻³ mole) was suspended in 250 ml of absolute EtOH. Then, an ethanolic solution of HCl (1.5 eq) was added. The reaction mixture was warmed (45° C.) until a clear solution was obtained, and a hot filtration of the clear solution was done. To this filtrate was added tert-butyl methyl ether (200 ml) to deposit a solid. Then the precipitate was filtered under vacuum and dried in oven at 65° C. for 24 hours to give 0.4 g of ondansetron hydrochloride Form H. KF=1.7%.

Preparation of Ondansetron Hydrochloride Form I

Example 39: Ondansetron hydrochloride Form I was prepared by treating hydrated or anhydrous ondansetron hydrochloride in methanol vapors for three weeks at room temperature. The procedure was as follows: A 100-200 mg sample of ondansetron hydrochloride Form A or anhydrous ondansetron hydrochloride was kept in a 10 ml open glass bottle. The open bottle was placed in a larger bottle containing few milliliters of methanol. The larger bottle was sealed in order to create a saturated atmosphere. Following two weeks, the resulting solid was analyzed by X-Ray diffraction without further treatment, and found to be ondansetron hydrochloride Form I.

Preparation of Ondansetron Anhydrous Form B from Ondansetron Base

Example 40: Ondansetron base (10 g, 34.1 mmol, 1 eq.), 250 ml absolute ethanol and 8.4 ml of 23.3% HCl in ethanol (51.2 mmol, 1.5 eq.) were added to a 500 ml round bottle flask equipped with a calcium chloride tube and a mechanical stirrer. The mixture was stirred at room temperature for 66 hours. The solid was then filtered, washed with absolute ethanol (2×20 ml) and dried at 65° C. for 20 hours to obtain 8.7 g (77%) of ondansetron hydrochloride Form B, KF=0.66%.

Example 41: Ondansetron base (10 g. 34.1 mmol, 1 eq.), 250 ml absolute ethanol and 8.4 ml of 23.3% HCl in ethanol (51.2 mmol, 1.5 eq.) were added to a 500 ml round bottle flask equipped with a calcium chloride tube, a mechanical stirrer and a condenser. The mixture was heated to reflux to obtain a clear solution for about 30 min. The reaction mixture was then cooled to room temperature during which time a precipitation was formed. The reaction mixture was stirred for an additional 45 hours. The solid was then filtered, washed with absolute ethanol (2×20 ml) and dried at 65° C. for 20 hours to obtain 8.5 g (76%) of ondansetron hydrochloride Form B, KF=0.34%.

We claim:

1. Ondansetron hydrochloride monohydrate.

2. Ondansetron hydrochloride monohydrate containing about 5% water.

3. The ondansetron hydrochloride monohydrate of claim 1 characterized by a powder X-ray diffraction pattern having a strong peak at 23.3±2 degrees two-theta.

4. The ondansetron hydrochloride monohydrate of claim 3 further characterized by peaks in the powder X-ray diffraction pattern at 6.1, 12.4, 17.0, 18.3, 19.2, 20.3, 20.9, 24.1, 25.8, 28.1 and 30.3±0.2 degrees two-theta.

5. A process for preparing the ondansetron hydrochloride monohydrate of claim 1 comprising the steps of:
a) contacting crystals of ondansetron hydrochloride dihydrate with a mixture of from about 4% to about 50% water in ethanol,
b) separating the ethanol:water mixture, and
c) recovering the crystals as ondansetron hydrochloride monohydrate.

6. The process of claim 5 wherein the contacting occurs at the reflux temperature of the ethanol:water mixture.

7. The process of claim 5 wherein the dihydrate and monohydrate are denominated Form A expressing that their crystal structures are the same.

8. A process for preparing ondansetron hydrochloride dihydrate Form A comprising the steps of:
a) providing crystals of the ondansetron hydrochloride monohydrate of claim 1,
b) hydrating the crystals under an atmosphere of 50% relative humidity or greater, and
c) collecting the hydrated crystals containing about 10% water of crystallization.

9. Ondansetron hydrochloride Form A containing between about 5% water and 10% water.

10. A process for preparing the ondansetron hydrochloride Form A of claim 9, comprising the steps of:
a) suspending ondansetron free base in a liquid medium selected from the group consisting of absolute ethanol, a mixture of ethanol and isopropanol, and chloroform,
b) dissolving the free base by adding anhydrous HCl to the suspension,
c) crystallizing ondansetron hydrochloride from the liquid medium, and
d) separating the crystals from the liquid medium.

11. The process of claim 10 wherein the liquid medium is absolute ethanol.

12. The process of claim 10 wherein the HCl is added in an amount of 1±0.1 equivalent with respect to the ondansetron free base.

13. The process of claim 10 wherein the anhydrous HCl is added as a gas.

14. The process of claim 10 wherein the anhydrous HCl is added in solution in an inert organic solvent.

15. The process of claim 10 wherein the absolute ethanol is heated to hasten the dissolution of the ondansetron free base.

16. A process for preparing the ondansetron hydrochloride Form A of claim 9 comprising the steps of:
a) dehydrating crystals of ondansetron hydrochloride dihydrate by contacting with a liquid medium selected from the group consisting of ethanol, mixtures of ethanol and water, toluene and mixtures of ethanol and toluene,
b) separating the liquid medium from the crystals, and
c) collecting the crystals.

17. The process of claim 16 wherein the crystals are mechanically agitated during dehydration.

18. The process of claim 17 wherein the mechanical agitation is sonication.

19. Anhydrous ondansetron hydrochloride.

20. Anhydrous ondansetron hydrochloride Form B

21. Ondansetron hydrochloride Form B characterized by powder X-ray diffraction peaks at 10.5, 11.9, 13.0, 13.5, and 15.1±0.2 degrees two-theta.

22. Ondansetron hydrochloride Form B characterized by powder X-ray diffraction peaks at 10.5, 11.9, 10.5, 13.0, 13.5, 15.1, 20.9, 22.7, 24.0, and 25.7±0.2 degrees two-theta.

23. A pharmaceutical composition comprising the ondansetron hydrochloride of any of claims 1 through 22 and a pharmaceutically acceptable carrier.

24. A method for treating nausea and/or vomiting with the pharmaceutical composition of claim 23,

25. A process for preparing the ondansetron hydrochloride of any of claims 19 through 22 by treating ondansetron hydrochloride with a dry alcohol.

26. The process of claim 25 wherein the solvent is absolute ethanol.

27. The process of claim 25 wherein ondansetron hydrochloride that is treated with dry alcohol is Form A.

28. The process of claim 25 wherein the treatment is carried out at about 20° C.

29. The process of claim 28 wherein ondansetron hydrochloride that is treated with dry alcohol is Form A.

30. The process of claim 25 wherein the alcohol is ethanol, isopropanol, 1-butanol or a mixture of thereof.

31. The process of claim 30 wherein ondansetron hydrochloride that is treated with dry alcohol is Form A.

32. A process of preparing the ondansetron hydrochloride of any of claims 19 through 22 by treating ondansetron HCl in a dry organic solvent.

33. The process of claim 32 wherein the solvent is absolute ethanol.

34. The process of claim 32 wherein ondansetron hydrochloride that is treated with dry alcohol is Form A.

35. The process of claim 32 wherein the solvent is a ketone.

36. The process of claim 35 wherein ondansetron hydrochloride that is treated with dry alcohol is Form A.

37. The process of claim 32 wherein the treatment is carried out at about 20° C.

38. The process of claim 37 wherein ondansetron hydrochloride that is treated with dry alcohol is Form A.

39. Ondansetron hydrochloride Form B having a particle size below about 300 microns.

40. A pharmaceutical composition comprising the ondansetron hydrochloride Form B of claim 39 and a pharmaceutically acceptable carrier.

41. Ondansetron hydrochloride Form B having a particle size below about 200 microns.

42. A pharmaceutical composition comprising the ondansetron hydrochloride Form B of claim 41 and a pharmaceutically acceptable carrier.

43. Ondansetron hydrochloride Form B having a particle size below about 40 microns.

44. A pharmaceutical composition comprising the ondansetron hydrochloride Form B of claim 43 and a pharmaceutically acceptable carrier.

45. Anhydrous ondansetron hydrochloride Form B with a water content up to about 2%.

46. A process for preparation of ondansetron hydrochloride Form B comprising reacting HCl gas with a toluene solution of ondansetron base.

47. The process of claim 46 wherein the ondansetron hydrochloride is dissolved at the reflux temperature of toluene.

48. The process of claim 46 wherein gaseous hydrochloride is bubbled into the toluene solution of ondansetron.

49. Ondansetron hydrochloride Form C and hydrates thereof, characterized by powder X-ray diffraction peaks at 6.3 and 24.4±0.2 degrees two-theta and other peaks at 9.2, 10.2, 13.1 and 16.9±0.2 degrees two-theta.

50. Ondansetron hydrochloride Form C and hydrates thereof, characterized by powder X-ray diffraction peaks at 6.3, 9.2, 10.2, 13.1, 16.9 and 24.4±0.2 degrees two-theta.

51. A process for preparation of the product of claim 49 or 50 which comprises the steps of:
a) dissolving ondansetron base in ethanol,
b) adding an ethanolic solution of hydrochloride,
c) filtering, and
d) evaporating the mother liquor.

52. Ondansetron hydrochloride Form D and hydrates thereof, characterized by powder X-ray diffraction peaks at 8.3, 14.0, 14.8 and 25.5±0.2 degrees two-theta.

53. A process for preparing the ondansetron hydrochloride Form D and hydrates thereof of claim 52 comprising the steps of:
a) melting ondansetron hydrochloride in the presence of xylene; and
b) adding the melt to ethanol.

54. The process of claim 53 wherein ondansetron hydrochloride Form A is melted in the presence of xylene.

55. The process of claim 53 wherein ethanol is at a temperature of from about −15° C. to about room temperature.

56. The process of claim 55 wherein the ethanol is at a temperature of about −10° C.

57. Ondansetron hydrochloride Form E and hydrates thereof, characterized by a strong powder X-ray diffraction peak at 7.4 degrees two-theta and other typical peaks at 6.3, 10.5, 11.2, 12.3, 13.0, 14.5, 15.9, 1 20.1, 20.8, 24.5, 26.2 and 27.2±0.2 degrees two-theta.

58. Ondansetron hydrochloride Form E and hydrates thereof, characterized by a strong powder X-ray diffraction peak at 7.4 degrees two-theta and other typical peaks at 6.3, 10.5, 11.2, 12.3, 13.0, 14.5, 15.9, 1 20.1, 20.8, 24.5, 26.2 and 27.2±0.2 degrees two-theta.

59. A process for preparation of the product of claim 57 or 58 which comprises the step of treating ondansetron hydrochloride in isopropanol.

60. The process of claim 59 wherein the ondansetron hydrochloride is Form A.

61. The process of claim 59 wherein the temperature of the isopropanol is from about room temperature to about reflux temperature.

62. Ondansetron hydrochloride isopropanolate.

63. Ondansetron hydrochloride Form E isopropanolate.

64. Ondansetron hydrochloride Form E mono-isopropanolate.

65. Ondansetron hydrochloride Form E hemi-isopropanolate.

66. Ondansetron hydrochloride Form E having a water content of up to about 10%.

67. Ondansetron hydrochloride Form H and hydrates thereof, characterized by powder X-ray diffraction peaks at 7.8, 14.0, 14.8 , 24.7 and 25.6±0.2 degrees two-theta.

68. A process for preparing the ondansetron hydrochloride Form H of claim 67 which comprises the steps of:
a) suspension of ondansetron base in absolute ethanol;
b) adding an ethanol solution of hydrochloric acid;
c) precipitating with the addition of ether; and
d) isolating the product.

69. The process of claim 68 wherein the ether is methyl tert-butyl ether or diethyl ether.

70. The process of claim 68 wherein the ether is dry.

71. A pharmaceutical composition comprising the ondansetron hydrochloride of any of claims 49, 50, 52, 57, 58 and 62-67 and a pharmaceutically acceptable carrier.

72. Ondansetron hydrochloride methanolate.

73. Ondansetron hydrochloride methanolate Form I.

74. Ondansetron hydrochloride Form I and hydrates thereof, characterized by a strong XRD peak at 25.0+0.2 degrees two-theta and other XRD peaks at 8.2, 9.3, 9.9, 11.1 and 24.9±0.2 degrees.

75. Ondansetron hydrochloride Form I and hydrates thereof, characterized by a strong XRD peak at 25.0±0.2 degrees two-theta and other XRD peaks at 8.2, 9.3, 9.9, 11.1, 13.9, 16.0, 17.0, 21.0, 22.6, 25.8, 27.3 and 28.0±0.2 degrees.

76. Ondansetron hydrochloride Form I and hydrates thereof, characterized by a strong XRD peak at 25.0±0.2 degrees two-theta and other XRD peaks at 6.9, 8.2, 8.7, 9.1, 9.3, 9.9, 11.1, 11.6, 13.8, 16.1, 16.9, 17.9, 21.1, 22.7, 25.7, 26.6, 27.4 and 27.9±0.2 degrees.

77. A process for crystallizing ondansetron hydrochloride Form I comprising exposing ondansetron hydrochloride to methanol vapor.

78. The process of claim 77 wherein the exposure is for a period of about three weeks or less.

79. The process of claim 77 wherein the exposure is at room temperature.

80. The process of claim 77 wherein ondansetron hydrochloride Form A is exposed to methanol vapor.

81. The process of claim 77 wherein ondansetron hydrochloride Form B is exposed to methanol vapor.

82. A process for preparing anhydrous ondansetron hydrochloride Form B comprising the steps of:
a) dissolving ondansetron base in absolute ethanol;
b) adding an ethanol/hydrochloric acid solution; and
c) filtering.

83. The process of claim 82 wherein the ethanol is substantially dry.

84. The process of claim 82 wherein the ondansetron base and the ethanol/hydrochloric acid solution are mixed at room temperature.

85. The process of claim 82 wherein the mixture of ondansetron base is heated to reflux temperature.

86. The process of claim 82 wherein the ondansetron base and the ethanol/hydrochloric acid solution are mixed for a period of about 30 to about 70 hours at room temperature.

87. Ondansetron hydrochloride with a particle size distribution of 100% particle size below about 100 microns.

88. Ondansetron hydrochloride with particle size distribution of 100% particle size below about 50 microns.

89. A pharmaceutical composition comprising ondansetron with a particle size distribution of 100% particle size below about 200 microns and a pharmaceutically acceptable carrier.

90. A pharmaceutical composition comprising ondansetron with a particle size distribution of 100% particle size below about 100 microns and a pharmaceutically acceptable carrier.

91. A pharmaceutical composition comprising ondansetron with particle size distribution of 100% particle size below about 50 microns and a pharmaceutically acceptable carrier.

92. A method for treating nausea and/or vomiting comprising the step of administering to a patient in need of such treatment a therapeutically effective amount of the pharmaceutical composition of claim 91.

93. A pharmaceutical composition containing ondansetron hydrochloride Form I and a pharmaceutically acceptable carrier.
